(19) United States
(12) Patent Application Publication
(10) Pub. No.: US 2026/0146999 A1
(43) Pub. Date: May 28, 2026

(54) TRANSTHYRETIN BIOMARKERS FOR DETECTING AND MONITORING CANCERS

(71) Applicant: Sun Jet Biotechnology Inc., Taipei City (TW)

(72) Inventor: Yeou-Guang TSAY, Taipei (TW)

(73) Assignee: Sun Jet Biotechnology Inc., Taipei City (TW)

(21) Appl. No.: 19/299,973

(22) Filed: Aug. 14, 2025

Related U.S. Application Data
(60) Provisional application No. 63/725,776, filed on Nov. 27, 2024.

(51) Int. Cl. G01N 33/574; G01N 33/68

(52) U.S. Cl. CPC G01N 33/57585; G01N 33/6893

(57) ABSTRACT

Disclosed is a method for diagnosing a cancer health state, or a change in cancer health state in a patient, or for diagnosing a risk of the change or presence of a cancer in a patient, comprising determining, in a plasma sample from said patient, one or more biomarker values that correspond to transthyretin-containing complex structures, and assigning the patient as having or not having cancer, or having or not having a change in cancer health state, or having or not having a risk of cancer based on said biomarker values, wherein said cancer is preferably lung cancer (LC).

### FIELD OF THE INVENTION

The present invention pertains to a biomarker and a use of biomarker based on transthyretin-containing complex structures for detecting and monitoring cancers.

### BACKGROUND OF THE INVENTION

Lung cancer is a type of malignant lung tumor that occurs when cell growth in the lung is uncontrolled. Carcinomas, which account for over 98% of lung cancers, are generally classified into two types based on the type of cell that initiates the tumor: small-cell lung carcinoma (SCLC) and non-small-cell lung carcinoma (NSCLC). Smoking is responsible for 85% of lung cancer cases, while other risk factors include genetic predisposition, exposure to radon gas, asbestos, or polluted air. SCLC is the more aggressive type of lung cancer, characterized by rapid spread, and the main risk factor is tobacco smoking. In contrast, NSCLC has been on the rise in recent years, particularly among non-smokers. The most common types of NSCLC are adenocarcinoma, squamous cell carcinoma, and large cell carcinoma. The former two originate from mucus-producing cells lining the smallest and larger airways, respectively, while the latter originates from neuroendocrine cells in the lung.

Over 80% of lung cancer (LC) patients who survive more than five years are those with early-stage disease, while high mortality is seen in patients with advanced disease. Although early-stage LC markers should improve prognosis, current clinical biomarkers are neither sensitive nor specific enough. Thus far, there are no FDA-approved plasma biomarkers for early LC detection. CEA (carcinoembryonic antigen) and CYFRA21-1 (cytokeratin-19 fragment) are biomarkers for lung cancer diagnosis and monitoring used by some hospitals. CEA is a glycoprotein that is normally produced during fetal development but is also expressed in the plasma in other types of cancers. High levels of CEA are most commonly found in cases of colorectal adenocarcinoma, but it is now considered an auxiliary biomarker for the monitoring or diagnosis of cancer due to its wide expression in multiple diseases. CYFRA21-1, a fragment of cytokeratin-19, is commonly found in epithelial cells, including those in the lung, and is more specific for squamous cell carcinoma than for other types of lung cancer. Nevertheless, various cancers or benign lung diseases may also cause an increase in CYFRA21-1. Therefore, these biomarkers are typically combined with other imaging methods.

Overall, there is still a pressing need for biomarkers with better all-around performance in LC screening. Protein complexes are the bona fide structures that harbor biological functions. In conventional western blotting, accurately quantifying these protein complexes is difficult due to transfer efficiency and operational inconsistencies.

### SUMMARY OF THE INVENTION

The inventors surprisingly found that different species of transthyretin complex structures (or multimeric structures) can be detected and quantified from human plasma sample, and that their levels and ratios are indicative of the risk of cancers including lung cancer (LC).

In one aspect, the present invention provides a method for diagnosing a cancer health state in a patient, comprising: determining, in a plasma sample from said patient, one or more biomarker values that correspond to transthyretin-containing complex structures; and assigning the patient as having or not having cancer, or having or not having a change in cancer health state, or having or not having a risk of cancer based on said biomarker values, wherein said cancer is preferably lung cancer (LC).

In one further aspect, the present invention provides a method for diagnosing a change in cancer health state in a patient, comprising: determining, in a plasma sample from said patient, one or more biomarker values that correspond to transthyretin-containing complex structures; and assigning the patient as having or not having cancer, or having or not having a change in cancer health state, or having or not having a risk of cancer based on said biomarker values, wherein said cancer is preferably lung cancer (LC).

In one yet aspect, the present invention provides a method for diagnosing a risk of the change or presence of a cancer in a patient, comprising: detecting, in a plasma sample from said patient, one or more biomarker values that correspond to transthyretin-containing complex structures; and assigning the patient as having or not having cancer, or having or not having a change in cancer health state, or having or not having a risk of cancer based on said biomarker values, wherein said cancer is preferably lung cancer (LC).

In one further aspect, the present invention provides use of a transthyretin-containing complex structure as a biomarker for cancer, including lung cancer (LC).

In one yet aspect, the present invention provides a capture reagent for transthyretin or a transthyretin-containing complex structure, for use in diagnosing (in vitro) a cancer health state in a patient, wherein said cancer is preferably lung cancer (LC).

Specifically, the use comprises determining one or more biomarker values that correspond to transthyretin-containing complex structures using the capture reagent for transthyretin or a transthyretin-containing complex structure.

Also provided is a kit for performing the method as described herein, comprising a capture reagent for transthyretin or a transthyretin-containing complex structure, and instructions for performing the method.

In some embodiments, the one or more biomarker values are determined by performing an in vitro assay. The in vitro assay may be an immunoassay, including but not limited to a western blotting and a capillary electrophoresis.

In some embodiments, determining the biomarker values comprises performing an in vitro assay, wherein said in vitro assay comprises a capture reagent for transthyretin or a transthyretin-containing complex structure.

In some embodiments, the capture reagent is an antibody.

In some embodiments, the one or more biomarker values are determined by performing a capillary electrophoresis under non-reducing conditions. According to certain preferred embodiments, the one or more biomarker values include IP₂₃₀, IP₃₂₀, IP₁₆₀, or a combination thereof. According to certain preferred embodiments, the assigning is based on a ratio of said biomarker values selected from the group consisting of

$$\frac{{IP}_{230}}{{IP}_{160}} , \frac{{IP}_{320}}{{IP}_{160}} and \frac{{IP}_{230} + {IP}_{320}}{{IP}_{160}} .$$

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of western blotting used to analyze plasma transthyretin species from three healthy subjects (HS) and nine lung cancer (LC) patients under non-reducing conditions. The numbers to the left mark the molecular masses (kDa) for the markers that migrated to these positions. Each of the three peaks characterized in capillary western blotting may contain the indicated structures detected using SDS-PAGE.

FIG. 2 shows the migration profiles of plasma transthyretin species in capillary western analyses for 31 healthy subjects and 86 lung cancer patients. All patients had a disease severity no higher than stage 2. The numbers to the left indicate percentages of signals relative to the height of peak P₁₆₀, while the numbers at the bottom denote the migration positions of proteins corresponding to their molecular masses. The pair of numbers at the top of the shaded box indicates the mass range boundaries used for summing signals.

FIG. 3 shows the box plot illustrating the statistical analysis of the transthyretin (TTR) indices in lung cancer patients and healthy subjects. The statistical analyses of three TTR multimeric indices were performed. Each dot in the plot represents the index value for an individual subject, with the plus sign (+) indicating the mean for each group.

FIG. 4 shows the receiver operating characteristic (ROC) curves of the TTR indices distinguishing between healthy individuals and lung cancer patients. Sensitivity is on the vertical axis, and (1−specificity) is on the horizontal axis for each TTR cutoff value. The dot in each plot corresponds to the point with the minimal Euclidean distance. The specific cutoff values are listed directly next to these dots, with corresponding performance indicated by dotted lines aligned with the axes.

FIG. 5 shows a summary of the results of three TTR indices for all healthy subjects (HS) and lung cancer patients in this study. Dark grey shading indicates values that are two times higher than the cutoff, whereas light grey shading represents values falling between one and two times the cutoff.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “biomarker” used herein refers to a measurable characteristic, either within or external to an organism, that indicates a specific physiological state or the presence of a disease. Biomarkers can be used as indicators for assessing physiological processes, disease progression, drug response, or treatment effectiveness. They may include molecules, cells, tissues, physiological indicators, or imaging features, with their changes often closely associated with disease occurrence, progression, treatment response, etc. Biomarkers have significant applications in clinical diagnosis, prediction, monitoring, and treatment, aiding in improving the accuracy of early disease detection, diagnosis, prognosis assessment, as well as evaluating the effectiveness and safety of treatment regimens.

The term “cancer” used herein refers to a group of diseases characterized by the uncontrolled growth and spread of abnormal cells. These cells can invade and destroy surrounding healthy tissues and can also metastasize to distant parts of the body. Cancer can arise from almost any type of cell in the body and may develop in various organs and tissues. It is typically caused by genetic mutations or other factors that disrupt the normal regulation of cell growth and division.

A biomarker value for the biomarkers described herein can be determined using any of a variety of known analytical methods. In some embodiments, the biomarker value can be determined through performing an in vitro assay, for example, an immunoassay. In one embodiment, the determination of a biomarker value involves the use of a capture reagent. A biomarker value may also refer to a ratio calculated based on two or more biomarker values, e.g.,

$$\frac{{IP}_{230}}{{IP}_{160}} , \frac{{IP}_{320}}{{IP}_{160}} and \frac{{IP}_{230} + {IP}_{320}}{{IP}_{160}} .$$

As used herein, a “capture agent” or “capture reagent” refers to a molecule that is capable of binding specifically to a biomarker. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, imprinted polymers, avimers, peptidomimetics, a hormone receptor, a cytokine receptor, and synthetic receptors, and modifications and fragments of these.

Transthyretin, encoded by the TTR gene, is one of the thyroid hormone-binding proteins responsible for the transport of thyroxine from circulation to target tissues. While the monomeric transthyretin has a molecular mass of about 16 kDa, it predominantly appears as a 55-kDa homotetramer due to non-covalent linkages (Zanotti G, Folli C, Cendron L, et al., FEBS J. 2008; 275 (23): 5841-5854). There is only one cysteine residue in the TTR sequence, and no intrachain or interchain disulfides have been reported. Based on the UniProt database, this residue can be oxidized to become cysteine sulfate. It is known that 40% of transthyretin interacts with retinol-binding proteins to form complexes, thereby participating in retinol transport and preventing its loss through glomerular filtration (Magalhães J, Eira J, Liz M A., Cell Mol Life Sci. 2021; 78 (17-18): 6105-6117). Our comparative platform for complex biomarkers showed that both transthyretin and retinol-binding proteins had significant increases in lung cancer patients. However, they were not co-eluted in the LC column and hence were not likely detected as heteromultimers (data not shown). Previous reports have revealed that, even if present in plasma, these complexes may become disrupted by solutions with high ionic strengths, such as those used in ion exchange chromatography.

While reflecting nutritional state and liver function, transthyretin levels may increase in diseases such as Hodgkin's granuloma and shigellosis, but decrease in conditions like malignancy, diabetes mellitus, hepatic cirrhosis, peritoneal dialysis, and inflammation. Using quantitative analyses for plasma transthyretin, it has been reported that TTR levels showed no significant change between LC patients and those with benign pulmonary diseases, and there was no cutoff that could effectively distinguish these two groups (Long T, Zhu X, Tang D, Li H, Zhang P, Int J Biol Markers. 2024; 39 (2): 130-140). Although total TTR amounts were documented in these studies, it was not addressed whether transthyretin complex structures changed in cancer patients.

The terms “transthyretin multimer,” “transthyretin-containing complex structure,” and “transthyretin complex structure” are used interchangeably herein and refer to a protein complex comprising at least one transthyretin subunit, wherein the at least one transthyretin subunit may be linked to one or more partners (proteins or polypeptides other than transthyretin).

In one aspect, the present invention provides a method for diagnosing a cancer health state, or a change in cancer health state in a patient, or for diagnosing a risk of the change or presence of a cancer in a patient, comprising determining, in a plasma sample from said patient, one or more biomarker values that correspond to transthyretin-containing complex structures, and assigning the patient as having or not having cancer, or having or not having a change in cancer health state, or having or not having a risk of cancer based on said biomarker values, wherein said cancer is preferably lung cancer (LC).

In another aspect, the present invention provides use of a transthyretin-containing complex structure as a biomarker for cancers, including LC.

The present invention also provides a capture reagent for transthyretin or a transthyretin-containing complex structure, for use in diagnosing (in vitro) a cancer health state in a patient. Said use may comprise determining one or more biomarker values that correspond to transthyretin-containing complex structures using the capture reagent for transthyretin or a transthyretin-containing complex structure.

In one further aspect, the present invention provides a kit for performing the method as described herein, comprising a capture reagent for transthyretin or a transthyretin-containing complex structure, and instructions for performing the method.

In one further aspect, the present invention provides use of a capture reagent for transthyretin or a transthyretin-containing complex structure in the preparation of a kit for performing the method as described herein.

According to the present invention, a patient may be assigned as having or not having a cancer, or having or not having a change in a cancer health state, or having or not having a risk of a cancer, based on a higher biomarker value that corresponds to a transthyretin-containing complex structure, or a lower biomarker value that corresponds to a transthyretin-containing complex structure.

As used herein, a higher (biomarker) value or lower (biomarker) value can refer to a value that is higher or lower compared with a reference level. For example, a lower value can be lower than a reference level by at least 30%, 40%, 50%, 60%, 70%, 80%, 90% or 100%; and higher value can be higher than a reference level by at least 30%, 40%, 50%, 60%, 70%, 80%, 90% or 100%. In some embodiments, a reference level can be a standard (or a threshold) value in a normal individual or a control group. For example, a standard or threshold value can be set based on an average or median level obtained from a cohort of normal subjects. In some embodiments, the cohort of subjects can be a population of normal human subjects (without cancer, or without a cancer). In addition, a threshold value can be set further based on a desired sensitivity and/or specificity for detecting or diagnosing a cancer.

According to certain embodiments of the present invention, three species or groups of human plasma transthyretin-containing proteins can be resolved using conventional SDS-PAGE and western blotting under non-reducing conditions, including: (i) primary 245-kDa complexes (“P complexes”), (ii) high-MW group (“H complexes”) having bands containing structures with molecular weights ranging from 330 kDa to 430 kDa, and (iii) protein megacomplexes (“M complexes”) trapped in stacking gels, with molecular masses higher than 700 kDa.

According to the present invention, human plasma transthyretin complex structures can also be resolved by a capillary gel electrophoresis into three major peaks, including P₂₃₀, P₃₂₀ and P₁₆₀, and correspond to the H complexes, M complexes, and P complexes, respectively.

A biomarker value is indicative of a concentration of a biomarker, or a ratio of concentrations of the biomarkers in a sample. A biomarker value of the present invention may be a signal intensity or normalized signal intensity of any of the peaks P₂₃₀, P₃₂₀ and P₁₆₀, denoted as IP₂₃₀, IP₃₂₀, IP₁₆₀, respectively, or a ratio of the signal intensities. The signal intensity can be measured as the area under the peak. In some embodiments, the signal intensity is measured as the area under the peak in an immunodetection.

In some embodiments, the one or more biomarker values are determined through performing a capillary electrophoresis under non-reducing conditions. Specifically, biomarker signals are detected by performing the capillary electrophoresis and immunodetection, and then the one or more biomarker values are determined based on the detected biomarker signals. According to certain preferred embodiments, the one or more biomarker values include IP₂₃₀, IP₃₂₀, IP₁₆₀, or a combination thereof. According to certain preferred embodiments, based on a ratio of said biomarker values selected from the group consisting of

$$\frac{{IP}_{230}}{{IP}_{160}} , \frac{{IP}_{320}}{{IP}_{160}} and \frac{{IP}_{230} + {IP}_{320}}{{IP}_{160}} ,$$

the patient is assigned as having or not having a cancer, or having or not having a change in a cancer health state, or having or not having a risk of a cancer.

The present invention is further illustrated by the following examples, which are provided for the purpose of demonstration rather than limitation.

### Examples

### 1. Materials and Methods

### 1.1 Reagents

Immobilon Western Chemiluminescent HRP Substrate (Millipore; Cat. No. WBKLS0500; Lot No. 2222001) was used for signal development in western blotting.

### 1.2 Clinical Samples

Plasma samples from lung cancer (LC) patients (IRB: EC1120809-F-E) were collected from the National Health Research Institutes Biobank. The pathology distribution of LC patients was empirically set to reflect clinical occurrence rates: 60% adenocarcinoma, 20% squamous cell carcinoma, 15% small-cell carcinoma, and 5% combined for males; 85% adenocarcinoma, 5% squamous cell carcinoma, 5% small-cell carcinoma, and 5% combined for females. Blood samples were processed into plasma according to the guidelines of the National Health Research Institutes Biobank. The supernatant, representing the plasma fraction, was stored at −80° C. until further use.

### 1.3 Western Blot Analysis

For each well, 0.3 μL of plasma sample was mixed with SDS-PAGE sample dye, which consists of 0.04 M Tris-HCl (pH 6.8), 1 M glycerol, 0.05 M SDS, and bromophenol blue for non-reducing analyses. After incubation at 37° C. for 30 minutes, the sample mixture was loaded into wells of a 4% stacking/7%-12% bipartite separating Tris-based polyacrylamide gel. After SDS-PAGE, the proteins were transferred to a nitrocellulose membrane in CAPS buffer (0.02 M 3-(Cyclohexylamino)-1-propanesulfonic acid in 10% methanol, pH 11). The blot was blocked with 1% BSA in TBST buffer (0.02 M Tris, 0.14 M NaCl, 0.1% Tween 20, pH 7.6) and then incubated with anti-transthyretin (GeneTex, GTX100577, rabbit-derived, diluted 1:3,000) overnight at 4° C., followed by anti-rabbit HRP conjugate (Jackson, donkey-derived, 711-035-152, diluted 1:10,000) for 1 hour at room temperature with TBST wash. Horseradish peroxidase-conjugated secondary antibodies were used to generate chemiluminescent signals, which were detected with the LAS-4000 (Fujifilm, Japan).

### 1.4 Automated Capillary Electrophoresis-Immunodetection

The reagents were purchased from Bio-Techne, USA, unless specified otherwise. Plasma samples were diluted 1:200, and 5× Fluorescent Master Mix was added to each sample. Incubation took place at 37° C. for 30 minutes. Four microliters of each sample were loaded into the top-row wells of plates preloaded with proprietary electrophoresis buffers designed for the separation of proteins ranging from 66 to 440 kDa. The other rows of the plate were filled with 1% bovine serum albumin (Bionovas, AA0530-0250; antibody diluent). The primary and secondary antibody solutions, chemiluminescence reagents, and wash buffer were utilized according to the manufacturer's instructions. For the biotinylated SimpleWestern size standard, these rows were filled with antibody diluent and streptavidin-HRP (GeneTex, GTX27403) instead of primary and secondary antibody solutions. The primary antibody used was anti-transthyretin (GeneTex, GTX100577, rabbit-derived, diluted 1:1,000). Anti-rabbit HRP conjugate (Jackson, donkey-derived, 711-035-152, diluted 1:2,000) was employed as the secondary antibody and was also diluted in the antibody diluent. The plates were centrifuged for 5 minutes at 1000 g at room temperature. The plates and capillaries were then loaded into a SimpleWestern™ system (Bio-Techne, USA), and assays were performed using the standard 66- to 440-kDa separation range protocol introduced in version 6.1.0 of the accompanying Compass software. The separation time was set to the default of 30 minutes, and the separation voltage was set at 475 V. Compass software reported data as chemiluminescence signals versus apparent molecular weight (MW). Apparent molecular weights (MW) were determined by aligning size marker peaks with capillary positions and utilizing signals from fluorescently labeled protein standards within the 5× Master Mix to compensate for variations in migration among capillaries.

### 1.5 Migration Profile Analysis

Analysis of data collected through capillary electrophoresis involved the use of Microsoft Excel 2021 Visual Basic for Applications (VBA). Raw data obtained from the Compass for SW software were converted to text format and transformed into graphs, with the x-axis representing log-scaled molecular weight and the y-axis representing the intensity of the immunodetection signals. To identify specific mass ranges, we employed an in-house peak detection and integration program, which was further validated through manual confirmation. These identified ranges were then utilized to sum the specified peak areas. The sensitivities and specificities for different cutoff values were characterized using receiver operating characteristic (ROC) curve analyses.

### 2. Results

2.1 Plasma Transthyretin Multimeric Species with Sizes of 300-430 and >700 kDa Showed Significant Increases Compared to the 245-kDa Species in LC Patients

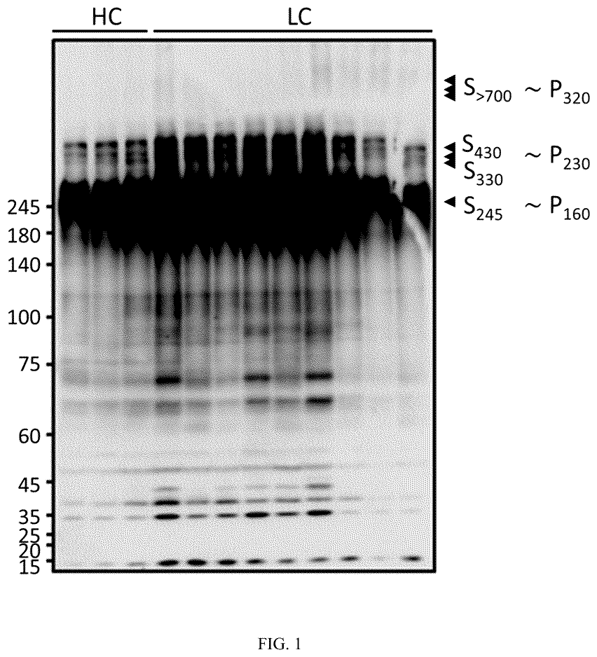

To profile the transthyretin multimeric structures present in human plasma, we conducted western blot analyses following conventional SDS-PAGE under non-reducing conditions. For healthy controls, a number of transthyretin-containing species with high molecular masses could be clearly discerned, with the ˜245-kDa structures showing the highest signals. Unexpectedly, the plasma level of the putative 15-kDa monomeric structure was quite low (FIG. 1).

The analysis of samples from LC patients showed that most of these transthyretin species were preserved. In addition to the primary 245-kDa complexes (P complexes), we also found two groups of TTR species whose signals increased markedly in lung cancer patients. The first high-MW group (H complexes) had bands containing structures with molecular weights ranging from 330 kDa to 430 kDa. The other group of protein megacomplexes (M complexes) was trapped in stacking gels, with molecular masses much higher than 700 kDa. In short, H and M complexes appear to have significant increases in most lung cancer patients (FIG. 1).

2.2 Peaks P₂₃₀ and P₃₂₀ Resolved Using Capillary Electrophoresis Showed Significant Increases Relative to the P₁₆₀ Species in LC Patients

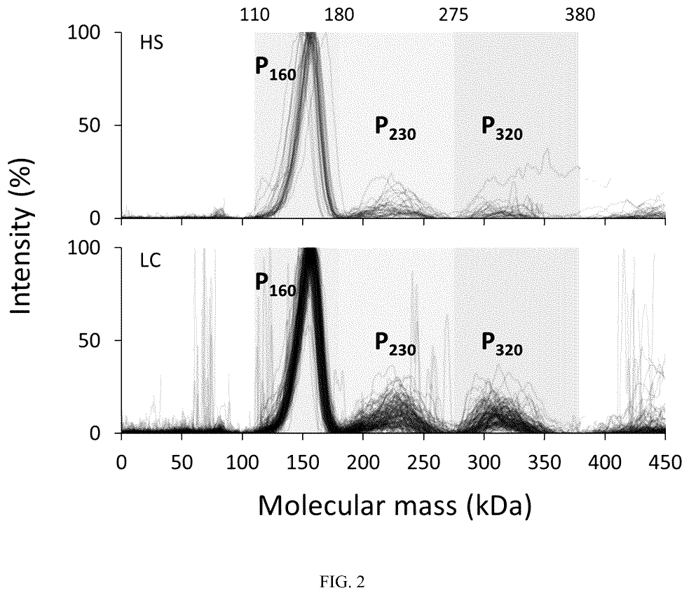

To quantitatively determine how transthyretin multimeric structures changed in cancer patients, we attempted to take advantage of the automated SimpleWestern system to analyze plasma samples under non-reducing conditions. This platform can resolve transthyretin structures into multiple peaks in a migration profile. In the profiles for healthy subjects, three peaks were discerned. The P₁₆₀ species had the highest signals in this system, likely corresponding to ˜245-kDa P complexes. Additionally, there were two other peaks, P₂₃₀ and P₃₂₀, in some of these subjects, likely equivalent to the H and M complexes revealed using SDS-PAGE (FIG. 1). For LC patients, with P₁₆₀ set as the reference, both peaks P₂₃₀ and P₃₂₀ appeared to increase significantly (FIG. 2). In order to quantitatively analyze these increases, we implemented three indices, including

$$\frac{{IP}_{230}}{{IP}_{160}} , \frac{{IP}_{320}}{{IP}_{160}} and \frac{{IP}_{230} + {IP}_{320}}{{IP}_{160}} ,$$

where IP₂₃₀, IP₃₂₀, and IP₁₆₀ are the signal intensities of these three peaks. Plasma samples from eighty-six LC patients, along with those from thirty-one healthy subjects, were subjected to SimpleWestern analyses. The median values of

$$\frac{{IP}_{230}}{{IP}_{160}} , \frac{{IP}_{320}}{{IP}_{160}} and \frac{{IP}_{230} + {IP}_{320}}{{IP}_{160}}$$

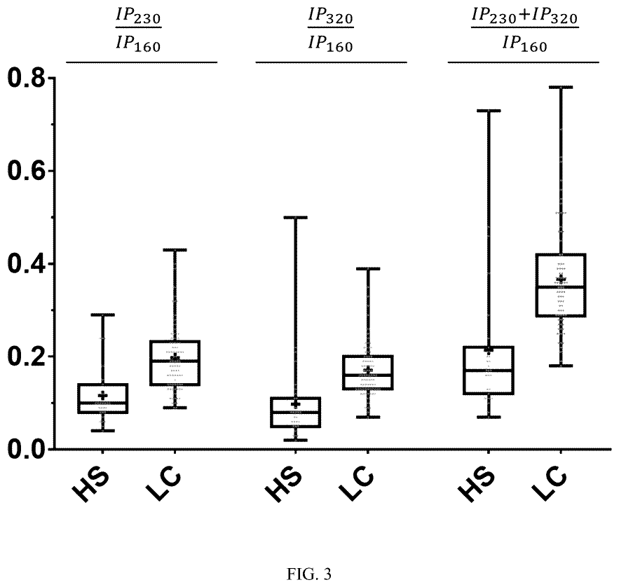

for healthy controls were 0.10, 0.08, and 0.17, respectively. For LC patients, the medians of these three indices increased to 0.19, 0.16 and 0.34. Overall, the means of these values were quite close to the medians (FIG. 3). Based on the boxplot, it seems that the index

$$\frac{{IP}_{230} + {IP}_{320}}{{IP}_{160}}$$

can best distinguish patients from healthy subjects.
2.3 all Three Transthyretin Multimeric Indices Performed Well in Distinguishing LC Patients from Healthy Controls

We next used ROC curves to investigate whether it is possible to find cutoffs for these three indices to distinguish patients from healthy controls. Overall, all of these curves are closer to the perfect classifier points than to the random classifier lines. The

$$\frac{{IP}_{320}}{{IP}_{160}} and \frac{{IP}_{230} + {IP}_{320}}{{IP}_{160}}$$

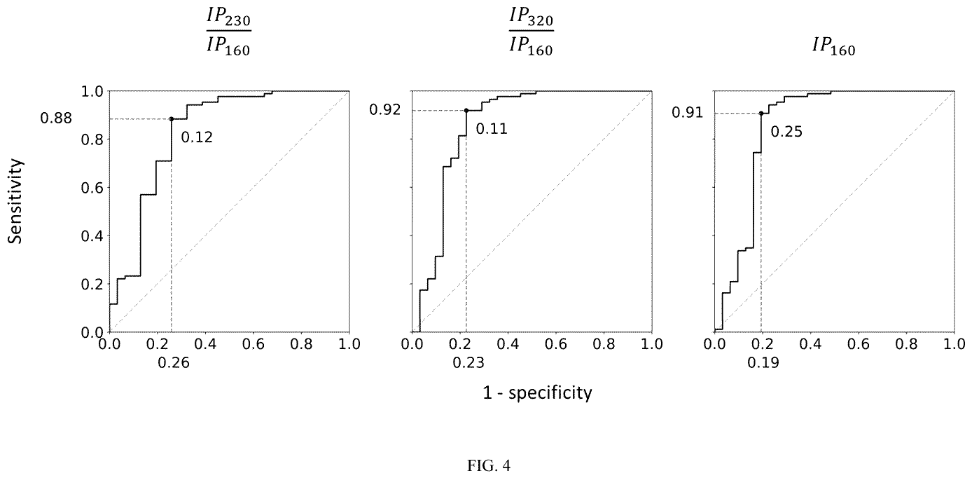

showed better performance in identifying LC patients with a sensitivity of 92% and 91%, respectively, and a specificity of 77% and 81% (FIG. 4).

We then explored the capacity of transthyretin indices in the screening of LC patients. With the ROC curves, we set the cutoff for

$$\frac{{IP}_{230}}{{IP}_{160}} at 0.12 and \frac{{IP}_{320}}{{IP}_{160}} at 0.11 ,$$

as well as that for

$$\frac{{IP}_{230} + {IP}_{320}}{{IP}_{160}}$$

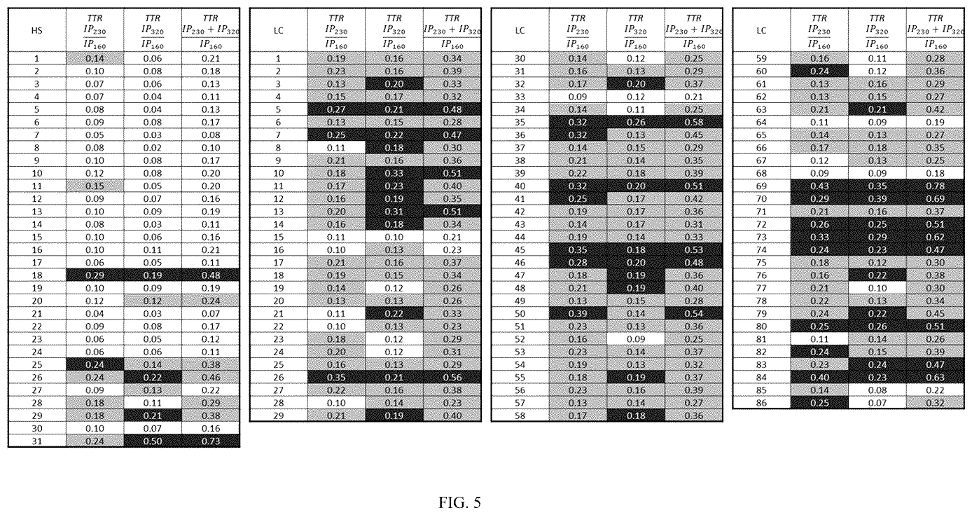

was 0.25 (FIG. 4) Using these standards, we can identify 95% of LC patients at stage I or II (FIG. 5). Overall, these results highlight the efficacy of these biomarker indices in high-sensitivity screening of cancer patients.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A method for diagnosing a cancer health state in a patient, comprising:
determining, in a plasma sample from said patient, one or more biomarker values that correspond to transthyretin-containing complex structures; and
assigning the patient as having or not having cancer, or having or not having a change in cancer health state, or having or not having a risk of cancer based on said biomarker values,
wherein said cancer is preferably lung cancer (LC).

2. The method of claim 1, wherein determining the biomarker values comprises performing an in vitro assay, wherein said in vitro assay comprises a capture reagent for transthyretin or a transthyretin-containing complex structure.

3. The method of claim 2, wherein the capture reagent is an antibody.

4. The method of claim 2, wherein the in vitro assay is a capillary electrophoresis under non-reducing conditions.

5. The method of claim 1, wherein the one or more biomarker values include IP₂₃₀, IP₃₂₀, IP₁₆₀, or a combination thereof.

6. The method of any one of claim 5, wherein the assigning is based on a ratio of said biomarker values selected from the group consisting of
$$\frac{{IP}_{230}}{{IP}_{160}} , \frac{{IP}_{320}}{{IP}_{160}} and \frac{{IP}_{230} + {IP}_{320}}{{IP}_{160}} .$$

7. A kit for performing a method for diagnosing a cancer health state in a patient, comprising a capture reagent for transthyretin or a transthyretin-containing complex structure, and instructions for performing the method, wherein said cancer is preferably lung cancer (LC).

8. The kit of claim 7 wherein the method comprising determining one or more biomarker values that correspond to transthyretin-containing complex structures using the capture reagent, and assigning the patient as having or not having cancer, or having or not having a change in cancer health state, or having or not having a risk of cancer based on said biomarker values.

9. The kit of claim 7, wherein the capture reagent is an antibody.

10. The kit of claim 8, wherein determining the biomarker values comprises performing an in vitro assay using the capture reagent.

11. The kit of claim 10, wherein the in vitro assay is a capillary electrophoresis under non-reducing conditions.

12. The kit of claim 7, wherein the one or more biomarker values include IP₂₃₀, IP₃₂₀, IP₁₆₀, or a combination thereof.

13. The kit of any one of claim 12, wherein the assigning is based on a ratio of said biomarker values selected from the group consisting of
$$\frac{{IP}_{230}}{{IP}_{160}} , \frac{{IP}_{320}}{{IP}_{160}} and \frac{{IP}_{230} + {IP}_{320}}{{IP}_{160}} .$$
